(19) United States
(12) Patent Application Publication
(10) Pub. No.: US 2026/0354724 A1
(43) Pub. Date: Nov. 12, 2026

(54) PHLEBOTOMY REVOLVER

(71) Applicant: Dominique Lockley, Henrico, VA (US)

(72) Inventor: Dominique Lockley, Henrico, VA (US)

(21) Appl. No.: 19/204,748

(22) Filed: May 12, 2025

(51) Int. Cl. A61B 5/155; A61B 5/15

(52) U.S. Cl. CPC A61B 5/155; A61B 5/150305; A61B 5/150343

(57) ABSTRACT

A phlebotomy device may comprise a housing and a rotatable carousel disposed within the housing. The carousel may be configured to hold a plurality of blood collection tubes. A vacutainer adapter may be coupled to the housing and may be configured to interface with a blood collection needle. A rotation mechanism may be coupled to the rotatable carousel and may be configured to rotate the carousel to sequentially position each of the plurality of blood collection tubes in fluid communication with the vacutainer adapter. A user input mechanism may be configured to receive a user input to activate the rotation mechanism. The device may include a control system to control the rotation mechanism based on the user input. Housing may comprise a movable section to allow loading and unloading of the blood collection tubes. A power source may be disposed within the housing and electrically coupled to the rotation mechanism.

### FIELD OF THE INVENTION

The present disclosure relates generally to medical devices and, more particularly, to devices and methods for collecting blood samples.

### BACKGROUND

Phlebotomy, the process of drawing blood from a patient for diagnostic or therapeutic purposes, is a common medical procedure. Conventional phlebotomy techniques typically involve manually switching blood collection tubes while a needle remains inserted in a patient's vein. This manual process may lead to several issues, including potential contamination due to incorrect order of draw, increased risk of hematoma formation, and the possibility of missing the vein during tube changes.

There is a need for improved devices and methods for collecting multiple blood samples that address these and other issues associated with conventional phlebotomy techniques.

### BRIEF OVERVIEW

The present disclosure describes various embodiments of a phlebotomy device, system, and method for collecting multiple blood samples. In some embodiments, the device may include a rotating mechanism for holding and automatically interchanging multiple blood collection tubes while maintaining a connection to a patient's bloodstream.

It should be appreciated that this Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Furthermore, the claimed subject matter is not limited to implementations that solve any or all disadvantages noted in any part of this disclosure.

### BRIEF DESCRIPTION OF THE DRAWINGS

The drawing figures depict one or more implementations in accord with the present teachings, by way of example only, not by way of limitation. In the figures, like reference numerals refer to the same or similar elements. Furthermore, it should be understood that the drawings are not necessarily to scale.

FIG. 1 is a perspective view of an exemplary phlebotomy revolver device, in accordance with some embodiments of the present disclosure.

FIG. 2 is a top view schematic diagram of internal components of an exemplary phlebotomy revolver device, in accordance with some embodiments of the present disclosure.

FIG. 3 is a perspective view of another exemplary phlebotomy revolver device, in accordance with some embodiments of the present disclosure.

### DETAILED DESCRIPTION

In the following detailed description, numerous specific details are set forth to provide a full understanding of the present disclosure. It will be apparent, however, to one ordinarily skilled in the art that embodiments of the present disclosure may be practiced without some of these specific details. In other instances, well-known structures and techniques have not been shown in detail so as not to obscure the disclosure.

The present disclosure is directed to a phlebotomy revolver device, system, and method for collecting multiple blood samples. The device may automate the process of interchanging blood collection tubes during a phlebotomy procedure, potentially reducing the risk of errors, contamination, and patient discomfort associated with conventional manual techniques.

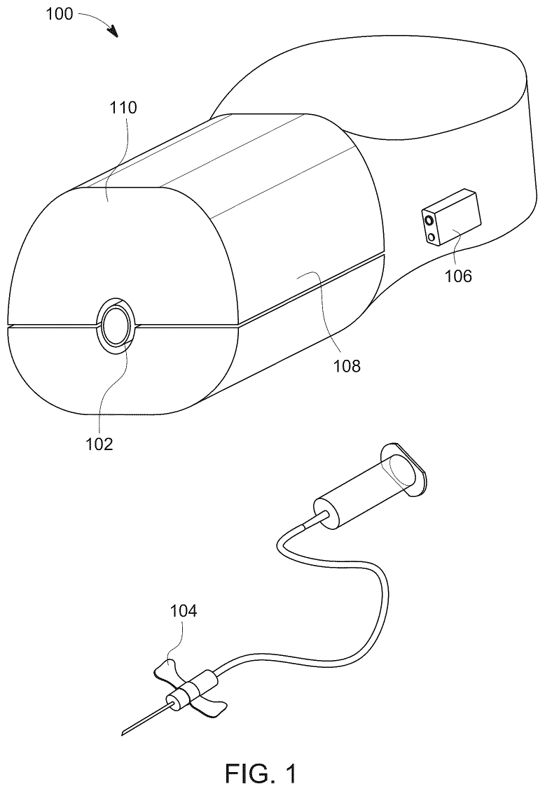

FIG. 1 illustrates an exemplary phlebotomy revolver device 100 according to some embodiments of the present disclosure. The device 100 may include a main body 102 that may be substantially cylindrical in shape. The main body 102 may house various internal components and may be designed for comfortable handheld use by a phlebotomist or other healthcare professional.

The device 100 may include a section 104 that may flip up to allow loading of blood collection tubes. This feature may provide easy access for inserting and removing tubes before and after the blood collection process. The device 100 may also include a vacutainer holder 106 on one end, which may be designed to interface with standard blood collection equipment such as butterfly needles or vacutainers.

In some embodiments, the device 100 may be battery-powered and capable of 360-degree rotation, as indicated by the label 108. This rotational capability may allow for automatic positioning of blood collection tubes during the phlebotomy procedure.

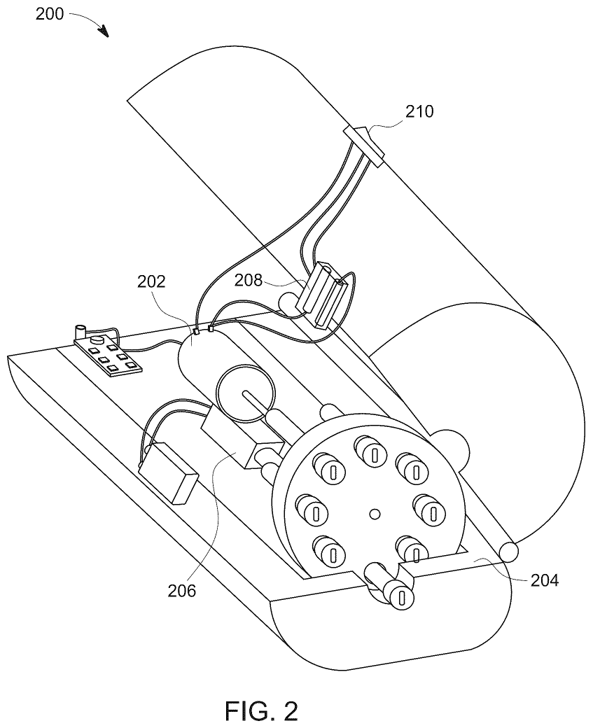

FIG. 2 provides a top view schematic diagram of internal components that may be included in some embodiments of the phlebotomy revolver device. These components may include a DC motor 202, which may drive the rotational mechanism of the device. A linear actuator 204 may be included to control precise movements of internal components.

The device may also include a vacutainer lid adapter 206, which may interface with blood collection tubes to maintain proper vacuum conditions during the collection process. A rechargeable battery 208 may provide power to the device, allowing for portable operation. An on/off switch 210 may be included to control the device's power state.

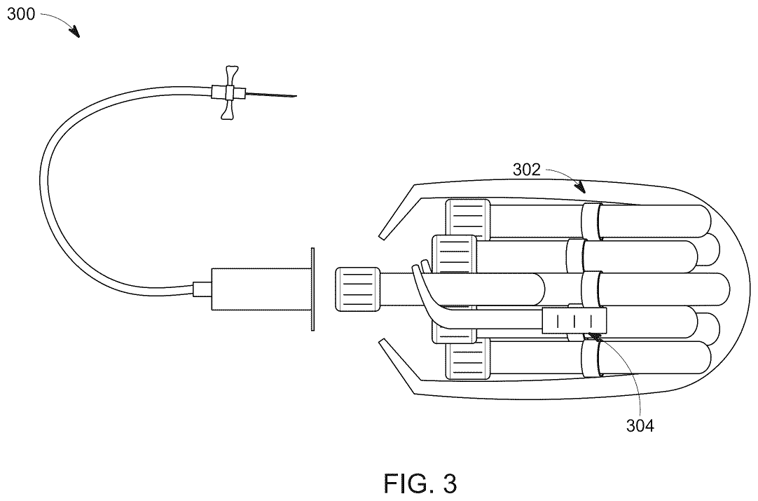

FIG. 3 illustrates another exemplary embodiment of a phlebotomy revolver device 300. This embodiment may include a revolving section 302, which may hold and rotate multiple blood collection tubes. A thumb push mechanism 304 may be included to allow the user to initiate rotation of the tubes with a simple press of the thumb.

In operation, a phlebotomist may load the device 100, 300 with the required number and type of blood collection tubes. The device may be connected to a patient's bloodstream via standard blood collection equipment attached to the vacutainer holder 106. The phlebotomist may then initiate the blood collection process.

As each tube is filled, the phlebotomist may activate the rotation mechanism, either through the thumb push 304 or another control mechanism. This may cause the device to rotate, automatically positioning the next empty tube for collection. This process may continue until all required samples have been collected.

The automated rotation and positioning of tubes may help maintain the correct order of draw, potentially reducing the risk of cross-contamination between samples. Additionally, by minimizing manual handling of tubes during the collection process, the device may help reduce the risk of needle movement, which could lead to hematoma formation or missed veins.

It should be understood that the above-described embodiments are merely possible examples of implementations, merely set forth for a clear understanding of the principles of the present disclosure. Many variations and modifications may be made to the above-described embodiments without departing substantially from the spirit and principles of the present disclosure. All such modifications and variations are intended to be included herein within the scope of this disclosure and protected by the following claims.

The phlebotomy revolver device may provide several technical advantages over conventional manual phlebotomy techniques. The automated rotation and positioning of blood collection tubes may help maintain the correct order of draw, potentially reducing the risk of cross-contamination between samples. By minimizing manual handling of tubes during the collection process, the device may help reduce the risk of needle movement, which could lead to hematoma formation or missed veins. This may improve patient comfort and safety during the blood collection procedure.

The device may also increase efficiency and accuracy of the phlebotomy process. By automating tube changes, the phlebotomist may be able to focus more on patient care and proper technique rather than manually switching tubes. This could potentially lead to faster blood collection times and reduced chances of human error in tube labeling or ordering.

In some embodiments, the phlebotomy revolver device may include additional features to further enhance its functionality. For example, the device may incorporate a barcode scanner or RFID reader to automatically identify and record information about each blood collection tube as it is positioned for filling. This may help ensure proper labeling and tracking of samples.

The device may also include a display screen that may provide real-time information to the phlebotomist, such as the current tube in use, the number of tubes filled, and any alerts or notifications. This may help the phlebotomist monitor the progress of the blood collection procedure more easily.

In some embodiments, the phlebotomy revolver device may be designed with interchangeable carousel modules to accommodate different sizes or types of blood collection tubes. This modular design may allow for greater flexibility in various clinical settings and for different types of blood tests.

The device may also incorporate a safety mechanism that may prevent rotation of the carousel while a needle is engaged with a blood collection tube. This may help prevent accidental needle dislodgement or patient injury during tube changes.

In alternative embodiments, the phlebotomy revolver device may be designed for use with different types of blood collection equipment beyond standard vacutainers. For example, it may be adapted for use with syringe-based blood collection systems or specialized pediatric blood collection devices.

The device may also be designed with ergonomic considerations to reduce physical strain on the phlebotomist during extended use. This may include features such as adjustable grips, balanced weight distribution, or the ability to be mounted on a stand or arm for hands-free operation.

In some embodiments, the phlebotomy revolver device may include wireless connectivity features. This may allow for integration with electronic health record systems, enabling automatic upload of blood collection data and reducing the need for manual data entry.

The device may also incorporate a self-diagnostic system that may alert the user to any malfunctions or required maintenance, helping to ensure consistent and reliable operation over time. The embodiments of the disclosure described above are intended to be merely exemplary; numerous variations and modifications will be apparent to those skilled in the art. All such variations and modifications are intended to be within the scope of the present disclosure as defined in any appended claims.

What is claimed is:

1. A phlebotomy device comprising:
a housing;
a rotatable carousel disposed within the housing and configured to hold a plurality of blood collection tubes;
a vacutainer adapter coupled to the housing and configured to interface with a blood collection needle;
a rotation mechanism coupled to the rotatable carousel and configured to rotate the carousel to sequentially position each of the plurality of blood collection tubes in fluid communication with the vacutainer adapter; and
a user input mechanism configured to receive a user input to activate the rotation mechanism.

2. The phlebotomy device of claim 1, wherein the user input mechanism comprises a thumb-actuated button.

3. The phlebotomy device of claim 1, further comprising a control system configured to control the rotation mechanism based on the user input.

4. The phlebotomy device of claim 1, wherein the housing comprises a section that is movable to allow loading and unloading of the blood collection tubes.

5. The phlebotomy device of claim 1, further comprising a power source disposed within the housing and electrically coupled to the rotation mechanism.

6. The phlebotomy device of claim 5, wherein the power source comprises a rechargeable battery.

7. The phlebotomy device of claim 1, wherein the rotation mechanism is configured to rotate the carousel through 360 degrees.

8. The phlebotomy device of claim 1, further comprising a linear actuator coupled to the rotation mechanism and configured to control precise movements of the rotatable carousel.

9. The phlebotomy device of claim 1, wherein the vacutainer adapter comprises a vacuumizer lid adapter configured to maintain vacuum conditions in the blood collection tubes during blood collection.

10. The phlebotomy device of claim 1, further comprising an on/off switch disposed on the housing and electrically coupled to the rotation mechanism.

11. A method of collecting multiple blood samples, the method comprising:
providing a phlebotomy device comprising a rotatable carousel configured to hold a plurality of blood collection tubes;
loading the plurality of blood collection tubes into the rotatable carousel;
connecting the phlebotomy device to a blood collection needle inserted into a patient;
collecting a blood sample in a first blood collection tube;
receiving a user input to rotate the carousel;
in response to the user input, rotating the carousel to position a second blood collection tube for blood collection; and
collecting a blood sample in the second blood collection tube.

12. The method of claim 11, wherein receiving the user input comprises detecting actuation of a thumb-actuated button on the phlebotomy device.

13. The method of claim 11, further comprising automatically maintaining a vacuum condition in each of the blood collection tubes during the blood collection.

14. The method of claim 11, further comprising repeating the steps of receiving a user input, rotating the carousel, and collecting a blood sample until blood samples have been collected in each of the plurality of blood collection tubes.

15. The method of claim 11, wherein rotating the carousel comprises electrically driving a rotation mechanism coupled to the carousel.

16. A phlebotomy system comprising:
a plurality of blood collection tubes;
a blood collection needle; and
a phlebotomy device comprising:
a housing,
a rotatable carousel disposed within the housing and configured to hold the plurality of blood collection tubes,
a vacutainer adapter coupled to the housing and configured to interface with the blood collection needle,
a rotation mechanism coupled to the rotatable carousel and configured to rotate the carousel to sequentially position each of the plurality of blood collection tubes in fluid communication with the vacutainer adapter, and
a user input mechanism configured to receive a user input to activate the rotation mechanism.

17. The phlebotomy system of claim 16, wherein the phlebotomy device further comprises a control system configured to control the rotation mechanism based on the user input.

18. The phlebotomy system of claim 16, wherein the housing of the phlebotomy device comprises a section that is movable to allow loading and unloading of the blood collection tubes.

19. The phlebotomy system of claim 16, wherein the phlebotomy device further comprises a power source disposed within the housing and electrically coupled to the rotation mechanism.

20. The phlebotomy system of claim 16, wherein the vacutainer adapter comprises a vacutainer lid adapter configured to maintain vacuum conditions in the blood collection tubes during blood collection.
